(19) United States
(12) Patent Application Publication
(10) Pub. No.: US 2011/0034794 A1
(43) Pub. Date: Feb. 10, 2011

(54) BLOOD GLUCOSE METER WITH INDEPENDENT VOICE IC SUPPORTING POST-PRODUCTION ADJUSTMENT OF VOICE FUNCTION

(76) Inventor: Chun-Yu Chen, Taipei City (TW)

Correspondence Address: Chen Chun-Yu, P.O.BOX 46-563, Taipei City 10499, Taipei, 104, TW

(21) Appl. No.: 12/854,903

(22) Filed: Aug. 12, 2010

(30) Foreign Application Priority Data
Aug. 4, 2009 (TW) 098214384

(51) Int. Cl. A61B 5/145

(52) U.S. Cl. 600/365

(57) ABSTRACT

A blood glucose meter with an independent voice IC supporting post-production adjustment of voice function is provided. The blood glucose meter is allowed to work with a replaceable voice carrier so as to provide voice services in different languages that help users to correctly and easily operate the blood glucose meter. The blood glucose meter features that the voice carrier is not built in the blood glucose meter during production of the blood glucose meter. Instead, a driving module is edited in an IC chip of the blood glucose meter. When an actual ordered amount is confirmed, the different voice carriers can be assembled to a voice-IC expansion port of the blood glucose meter, thereby reducing manufacturing and inventory costs for the blood glucose meter.

### BACKGROUND OF THE INVENTION

1. Technical Field

The present invention relates to a blood glucose meter with an independent voice IC that supports post-production adjustment of its voice function. More particularly, the subject matter has its voice carrier and the blood glucose meter manufactured separately and later assembled when a manufacturer confirms the actually required amount for voice carriers of different languages according to the market demand reflected in the received purchase orders, so as to reduce manufacturing and inventory costs for the blood glucose meter.

2. Description of Related Art

Diabetes is one of the modern lifestyle diseases. When insulin secretion in blood is insufficient, blood glucose can not be effectively converted into energy. The surplus blood glucose accumulates in human body may cause apoplexy and other diseases. A diabetic, even under a medication course, has to frequently monitor his/her blood glucose value to ensure the properness of his/her diet and exercise and control diabetic danger. Since diabetics are mainly middle-age and old-age people, a blood glucose meter is desired to support voice services in different languages so as to, by simple and clear voice navigation, help its users to use it more effectively and correctly.

Conventionally, in the production of an existing blood glucose meter, voice files and a voice driver therefor are edited and recorded in the same chip, which is then built in the blood glucose meter. In view of the market tending toward internationalization, the blood glucose meter is desired to support various languages for meeting consumers' expectation. However, to make blood glucose meters operate in other languages than the existing ones, the voice files and the voice drivers have to be reprogrammed, meaning that the chips have to be replaced. Consequently, increased cost is required by preparing the additional chips. Meantime, since the chips are usually made in batches, the considerable amount of the chips for different languages in turn brings difficulty in inventory control.

Hence, how to overcome the above-mentioned problems is the target aimed by the inventor of the present invention.

### SUMMARY OF THE INVENTION

In view of the aforementioned defects of the prior art, the inventor of the present invention has paid effort to collect related information, and perform comprehensive evaluation and consideration, and, with his years of experience in the art as well as repeated tests and modifications, finally invented a novel blood glucose meter with an independent voice IC supporting post-production adjustment of voice function.

The primary objective of the present invention is to provide the blood glucose meter with the independent voice IC, which is constructed by assembling two semi-finished products, namely a blood glucose meter and a voice carrier, so as to eliminate the need of maintain stock of blood glucose meters with many different-language voice ICs, and to eliminate the need of reediting voice files and voice drivers of different languages. As long as there are blood glucose meters and voice carriers for different languages in stock, the two parts can be assembled according to a received order, thereby reducing the inventory control costs.

The secondary objective of the present invention is to provide the blood glucose meter with the independent voice IC, wherein when an order is put for the blood glucose meter with the voice IC operating in a certain language, the production line can produce only the part out of stock, such as the voice carrier, and then assemble it with the part in stock, such as the blood glucose meter so as to prompt timely shipment caters for the market demands.

### BRIEF DESCRIPTION OF THE DRAWING

The invention as well as a preferred mode of use, further objectives and advantages thereof will be best understood by reference to the following detailed description of an illustrative embodiment when read in conjunction with the accompanying drawings, wherein:

FIG. 1 is a perspective view of the present invention;

FIG. 2 is an exploded view of the present invention;

FIG. 3 is a block diagram of the present invention;

FIG. 4 is an applied view of the present invention; and

FIG. 5 is another applied view of the present invention.

### DETAILED DESCRIPTION OF THE INVENTION

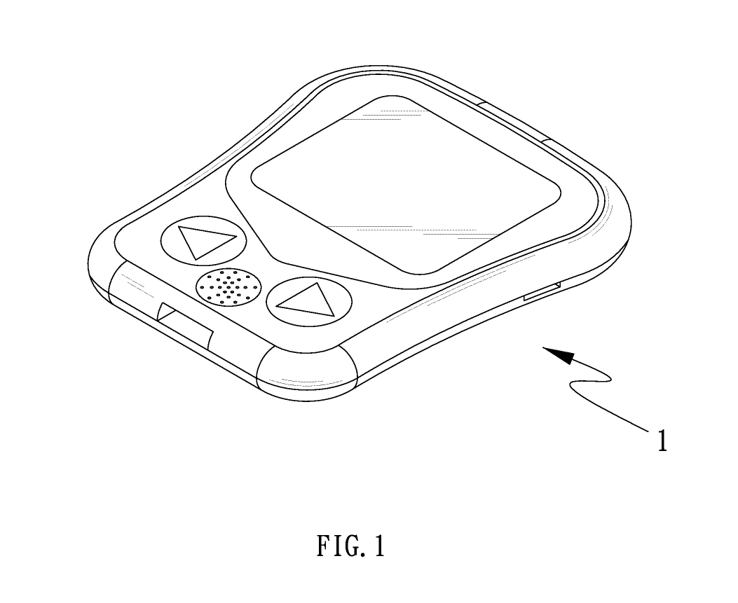

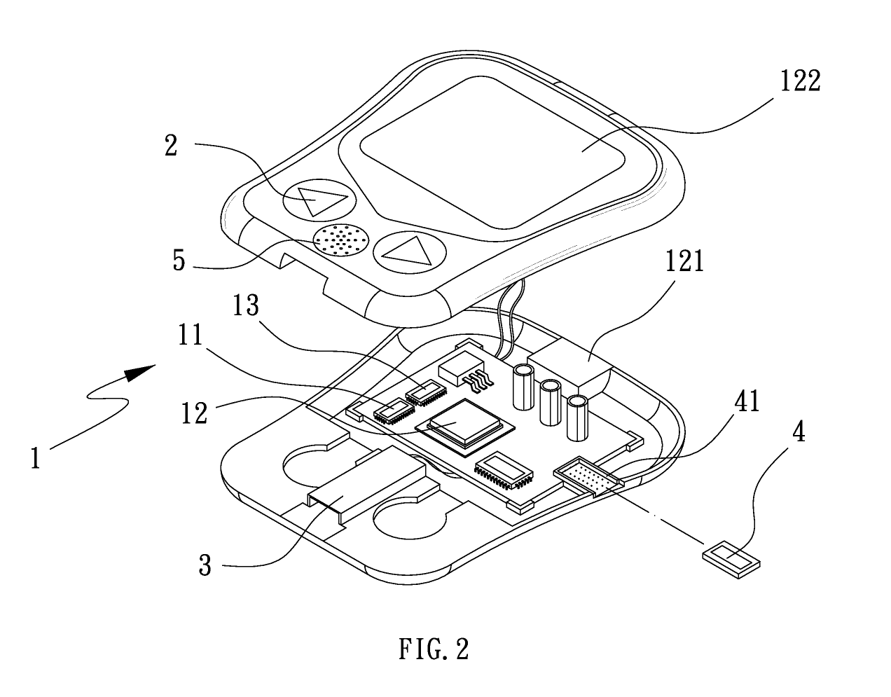

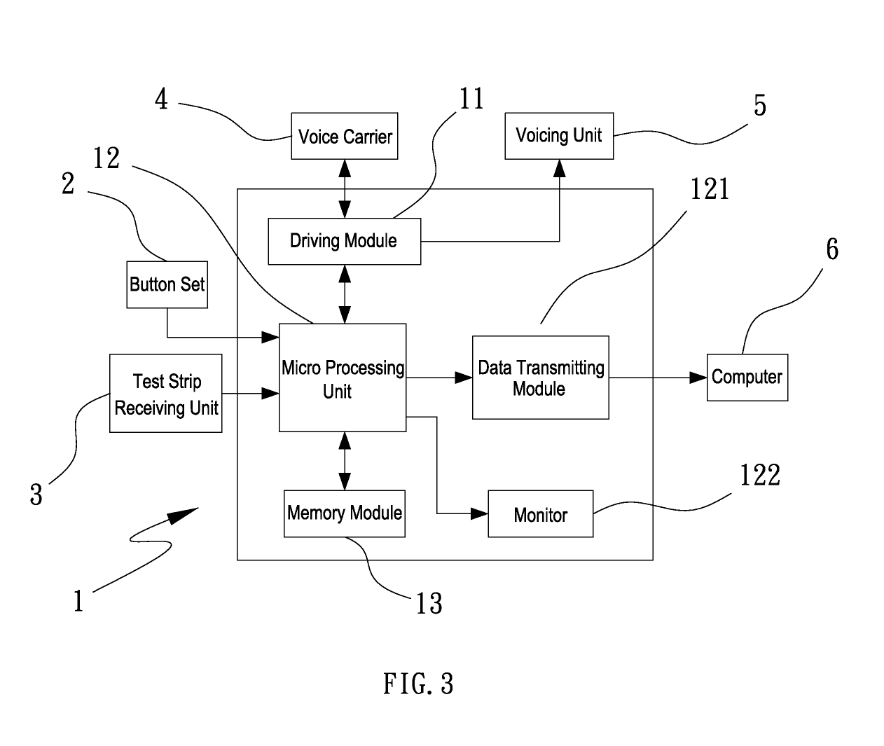

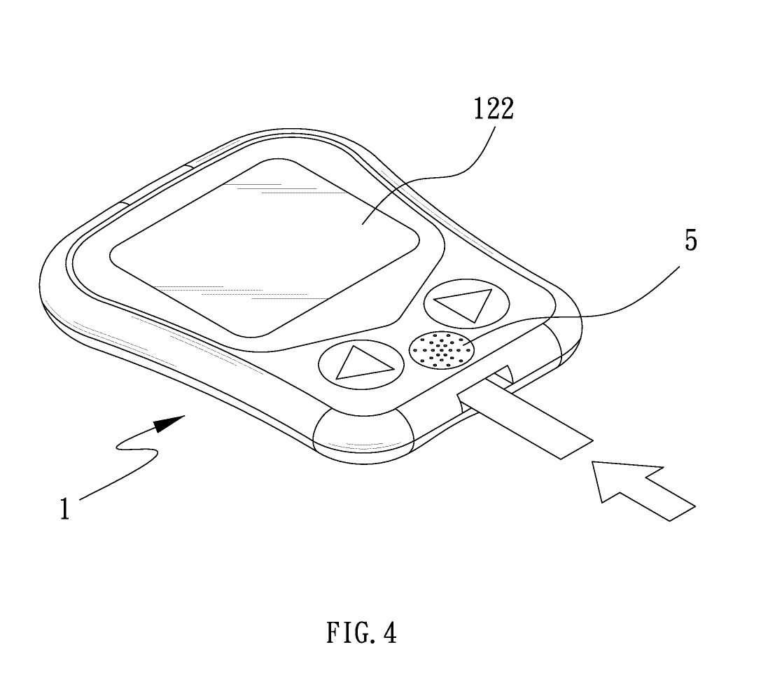

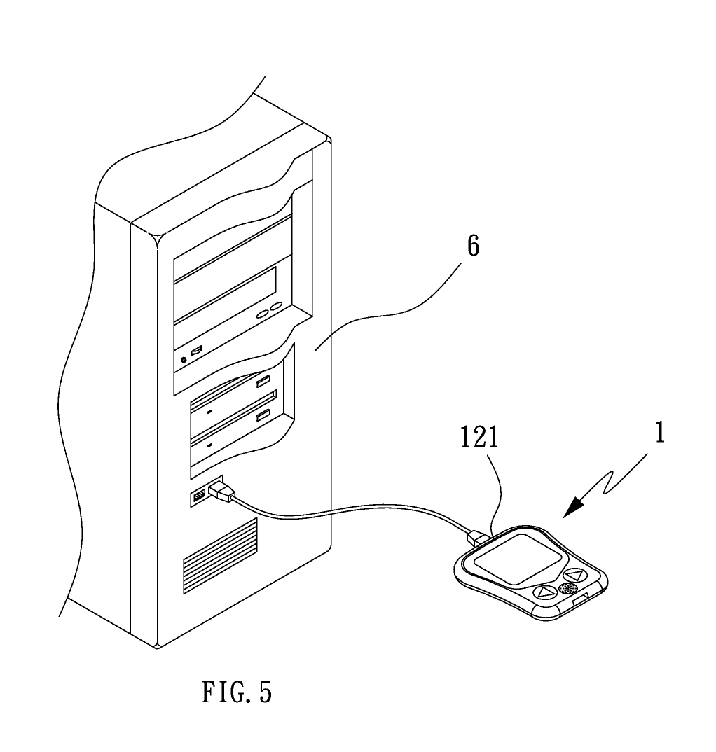

Please refer to FIG. 1, FIG. 2, FIG. 3, FIG. 4 and FIG. 5 for a perspective view, an exploded view, a block diagram and applied views of the present invention. As depicted, a blood glucose meter 1 with an independent voice IC supporting post-production adjustment of voice function primary comprises the following components:
(1) a button set 2 located at an exterior of the blood glucose meter 1 for being operated by a user to input personal information, to turn on power, to set date and time, and to modify the personal information;
(2) a test strip receiving unit 3 for collecting blood on a test strip and analyzing the blood, wherein the blood may be taken from a user by a lancing device and then combined with an enzyme preset on the blood glucose test strip for, such as, glucose oxidase examination, before delivered to the micro processing unit 12 for further blood analysis, in which the lancing device has its needle designed with an optimized cutting surface that protects skin texture from damage and supports automatic needle withdrawal, while the blood glucose test strip fits the blood glucose meter 1 in size and material;
(3) a micro processing unit 12 for processing and storing the analytic result of the test strip receiving unit 3 and for reading and automatically calibrating the data received by the test strip receiving unit 3 to further process and record the data;
(4) a driving module 11 for editing a voice driver required by the voice carriers 4 of different languages into a driving module 11 to be built in an IC, for driving the external voice carrier 4, wherein the voice driver may be edited by means of a programmable gate array, such as FPGA, or may be a ready-made voice driver;
(5) a voicing unit 5, as a known device, for giving out voice messages to assist a user in correctly operate the blood glucose meter 1;
(6) a voice-IC expansion port 41 for receiving the external voice carrier 4, so that a user can expand voice function for supporting a language he/she selects, wherein the voice carrier 4, as a detachable attached device inserted into the blood glucose meter, serves to provide voice files of different languages, in which one to three voice carriers 4 may be produced by the manufacturer, each supporting one to six languages, wherein the voice carrier 4 may be a flash memory or a ROM memory for recording therein voice files of certain languages;
(7) a large-area monitor 122 for clearly displaying values such as blood glucose value, date, time or remaining number of the test strips; and
(8) a data transmitting module 121 for, after use of the blood glucose meter, transmitting measured data to an external computer 6 for storage and update, wherein the data transmitting module 121 may be an infrared-ray emitter that transmits the data to the computer 6 through radio frequency or may be a USB device associated to the computer 6 for transmitting the blood glucose value to a blood-glucose management software, so as to provide detailed diagrams about blood glucose variation.

In operation, the test strip is inserted into the blood glucose meter 1, and related information is input to the micro processing unit 12 by using the button set 2. Then the voicing unit 5 reads settings of the voice carrier 4 and actuate related voice function to assist a user in operating the blood glucose meter 1. On the other hand, the test strip receiving unit 3, after collecting blood, transmits data to the micro processing unit 12, so the update is recorded by a memory module 13 in the blood glucose meter 1. Comparison between the current measured result and previous measured result is then displayed through the monitor 122 and transmitted to the computer 6 through the data transmitting module 121 for further comparison and analysis.

The subject matter of the present invention features that the voice carrier 4 is not built in the blood glucose meter 1 during production of the blood glucose meter 1. When the actual ordered amount of the blood glucose meter is confirmed, the voice carriers 4 of different languages are assembled to the voice-IC expansion port 41 of the blood glucose meter according to the ordered amount, thereby reducing manufacturing and inventory costs for the blood glucose meter.

The present invention has been described with reference to the preferred embodiment and it is understood that the embodiment is not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A blood glucose meter with an independent voice IC supporting post-production adjustment of voice function, the blood glucose meter comprising:
a button set located on a panel of the blood glucose meter for being used to input a personal information of a user;
a test strip receiving unit for collecting and analyzing blood on a test strip;
a micro processing unit for processing and storing an analytic result of the test strip receiving unit;
a voice-IC expansion port for receiving an external voice carrier so as to expand the voice function;
a driving module for editing a voice driver that supports voice carriers of different languages into a driving module that is then built on an IC for reading the external voice carrier;
a voicing unit located on the panel of the blood glucose meter for giving out voice to assist the user in operating the blood glucose meter correctly; and
a monitor located on the panel of the blood glucose meter for displaying a blood glucose value, a date, a time or a number of remaining test strips,
wherein the blood glucose meter is characterized in that the voice carrier is configured to be assembled to the blood glucose meter after the blood glucose meter is produced and the driving module is built in the blood glucose meter.

2. The blood glucose meter with the independent voice IC of claim 1, further comprising a data transmitting module for transmitting data related to measurement of the user who has used the blood glucose meter to an external computer for storing and updating purposes.

3. The blood glucose meter with the independent voice IC of claim 1, wherein each said voice carrier carries 1 to 6 languages for option.

4. The blood glucose meter with the independent voice IC of claim 2, wherein the data transmitting module is configured to transmit the data to the external computer through infrared ray, RFID, Bluetooth, USB or other wired or wireless transmission means.

5. The blood glucose meter with the independent voice IC of claim 1, wherein the blood glucose meter has plural said voice carriers.
